(19) United States
(12) Patent Application Publication
(10) Pub. No.: US 2018/0344597 A1
(43) Pub. Date: Dec. 6, 2018

(54) PRE-WASH COMPOSITION FOR CLEAN BENEFIT

(71) Applicant: The Procter & Gamble Company, Cincinnati, OH (US)

(72) Inventors: Supriya Punyani, Singapore (SG); Jun Ji Zhang, Singapore (SG); Shikhar Gupta, Singapore (SG); Stevan David Jones, Cincinnati, OH (US); Howard David Hutton, III, Oregonia, OH (US)

(21) Appl. No.: 16/000,290

(22) Filed: Jun. 5, 2018

Related U.S. Application Data
(60) Provisional application No. 62/515,131, filed on Jun. 5, 2017.

(51) Int. Cl. A61K 8/34; A61K 8/37; A61K 8/39; A61K 8/31; A61Q 5/00

(52) U.S. Cl. CPC A61K 8/345; A61K 8/37; A61Q 5/008; A61K 8/31; A61K 8/342; A61K 8/39

(57) ABSTRACT

The present invention discloses a pre-wash composition having from about 0.1% to about 12.0% of a sebum modifier material or mixture of sebum modifier materials, wherein the sebum modifier material has the following physical properties: i.) Molecular charge surface area higher than −8; and ii.) Shadow length greater than 18; from about 88 wt. % to about 99.9 wt. % water carrier; and from about 0.1 wt. % to about 5 wt. % emulsifier selected from the group consisting of anionic, non-ionic, cationic and amphoteric.

### FIELD OF THE INVENTION

The present invention relates to a pre-wash composition comprising one or more sebum modifiers, that is, materials that modify one or more sebum physical properties such as melting (and/or viscosity) of sebum and leads to superior cleaning when followed by shampoo. In addition, the present invention relates to a method of cleansing hair and scalp comprising applications and spreading on hair and scalp of a pre-wash composition comprising one or more sebum modifiers, that is, materials that modify one or more sebum physical properties such as melting (and/or viscosity) and leads to superior cleaning when followed by shampoo

### BACKGROUND OF THE INVENTION

Clean Scalp and Hair is described by consumer as having no sticky or greasy feel, no clumped fibers, no odor, and no weigh down of hair. Generally, consumers perceive dirty/unclean scalp and hair when sebum builds up on their scalp and their hair. The liquid sebum on hair and scalp is often associated with unclean, greasy, oily and dirty look, feel and smell. Sebum is continuously secreted out of the sebaceous glands on scalp in liquid form. Due to dynamic environment (exposure to UV and microflora, sebum is unstable and its composition rapidly changes. As a result, it typically exists in more than one phase on the scalp and it gets transferred to hair during the day. Most consumers use surfactant-containing shampoos to clean their hair. Cleansing with surfactant-containing shampoo, removed approximately 60-90% of the accumulated sebum, depending on the surfactant concentration. Most of the shampoos remove on average 80% of the sebum. It is perceived that many shampoos with high surfactant content strip the hair fiber surface from natural lubricants, leading to dry and squeaky hair feel. Thus, there is a need to develop hair cleansing products that effectively remove sebum. The inventors of the present invention surprisingly found that treating the hair with pre-wash compositions comprising materials that modify sebum physical properties, reducing its melting point and/or viscosity, significantly improves sebum removal by subsequent use of a shampoo product. The pre-wash composition can be applied, spread on the hair and scalp and rinsed off before shampooing. Alternatively, the pre-wash composition can be applied, spread on the hair, followed by application of shampoo and rinsing.

### SUMMARY OF THE INVENTION

The present invention is directed to a pre-wash composition for clean hair and scalp benefit comprising:
1) from about 0.1% to about 12.0% of a sebum modifier material or mixture of sebum modifier materials, wherein the sebum modifier material has the following physical properties:
   a. Molecular charge surface area higher than −8; and
   b. Shadow length greater than 18.
2) from about 88 wt. % to about 99.9 wt. % water carrier;
3) from about 0.1 wt. % to about 5 wt. % emulsifier selected from the group consisting of anionic, non-ionic, cationic and amphoteric
   The present invention is also directed to a cleansing method comprising the steps of
a. Applying on hair a pre-wash composition comprising:
   (1) from about 0.1% to about 12.0% of a sebum modifier material or mixture of sebum modifier materials wherein the sebum modifier material has the following physical properties:
      (a) Molecular charge surface area higher than -8; and
      (b) Shadow length greater than 18.
   2) from about 88 wt. % to about 99.9 wt. % water carrier;
   3) from about 0.1 wt. % to about 30 wt. % emulsifier selected from the group consisting of anionic, non-ionic, cationic and amphoteric
b. spreading the pre-shampoo on hair and scalp;
c. Optional rinsing the pre-wash composition from hair and scalp with water;
d. applying a shampoo composition on hair comprising from about 5 wt. % to about 40 wt. % of detersive surfactant;
e. spreading the shampoo composition on hair and scalp;
f. rinsing the shampoo composition from hair and scalp with water.
   wherein the cleansing method removes from about 10 wt. % to about 60 wt. % more sebum compared to a cleansing method that does not include steps a to c.

In a further embodiment, the cleansing method removes from about 15 wt. % to about 40 wt. % more sebum compared to a cleansing method that does not include steps a to c. In a further embodiment, the cleansing method removes from about 15 wt. % to about 30 wt. % more sebum compared to a cleansing method that does not include steps a to c.

The following classes of materials can reduce melting temperature of sebum.
a) branched aliphatic alcohols contains less than 20 total carbon atoms e.g. 2-hexyl decanol
b) unsaturated fatty acid contains less than 20 total carbon atoms e.g. Oleic acid c) a monoalkyl or dialkyl substituted glycerin or mono- or di-esters of glycerin with fatty acids wherein the structure of such monoalkyl- or dialkyl-substituted glycerin or glycerin esters contains less than 20 total carbon atoms;
d) an alkyl-substituted glycol wherein the structure of such alkyl substituted glycol contains less than 10 carbon atoms; e.g. propylene glycol long aliphatic hydrocarbons with greater than equal to 11 carbons e.g. Isododecane, C11-C12 Isoparaffin
The method of the present invention provides excellent cleaning performance without negatively affecting hair feel. The sebum modifier that is included in the pre-wash composition helps in the effective removal of sebum by a shampoo. Certain material can serve as sebum modifiers by reducing sebum's melting temperature (and/or reducing sebum viscosity). Without being limited by theory, the effective removal of sebum from hair and scalp may be caused by the easier emulsification of the sebum by the shampoo surfactant. Significant clean benefit is measured on hair treated by materials that caused significant sebum removal versus control hair that is not treated with such materials. The treatment involved the application of a 1% w/w solution of the materials in a mixture of water, emulsifier and thickner. Preferred materials include 2-hexyldecanol, oleic acid, Isododecane, isoparaffin C11-C12, PEG-7-cocoate. The cleaning is measured by amount of sebum removal using sebum fluorescence image analysis method. These and additional features provided by the embodiments of the present invention will be more fully understood in view of the following detailed description.

### DETAILED DESCRIPTION OF EMBODIMENTS OF THE INVENTION

All percentages and ratios used herein are by weight of the total composition, unless otherwise designated. All measurements are understood to be made at ambient conditions, where “ambient conditions” means conditions at about 25 ° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Leave-on,” in reference to compositions, means compositions intended to be applied to and allowed to remain on the keratinous tissue. These leave-on compositions are to be distinguished from compositions, which are applied to the hair and subsequently (in a few minutes or less) removed either by washing, rinsing, wiping, or the like. Leave-on compositions exclude rinse-off applications such as shampoos, rinse-off conditioners, facial cleansers, hand cleansers, body wash, or body cleansers. The leave-on compositions may be substantially free of cleansing or detersive surfactants. For example, “leave-on compositions” may be left on the keratinous tissue for at least 15 minutes. For example, leave-on compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

“Soluble” means at least about 0.1 g of solute dissolves in 100 ml of solvent, at 25° C. and 1 atm of pressure.

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M·Wt.” as used herein refers to the weight average molecular weight unless otherwise stated. The weight average molecular weight may be measured by gel permeation chromatography. “QS” means sufficient quantity for 100%.

The term “substantially free from” or “substantially free of as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

### “Pre-Wash Aqueous Composition”

“Pre-Wash”, in reference to compositions of the current invention means compositions intended to be used for applying the product before shampooing the hair. The pre-wash step may be followed by rinsing, wiping, of the like before shampooing. The pre-wash compositions may be substantially free of cleansing or detersive surfactants. For example, “Pre-wash compositions” may be left on the keratinous tissue for at least 2 minutes. For example, Pre-wash compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

The mechanism of action for sebum modifier involves changing the viscosity or the melting properties in order to enhance sebum removal.

In an embodiment of the present invention, the concentration of the Sebum Modifier Material or the concentration of the mixture of Sebum Modifier Material in a hair aqueous pre-wash composition is from about 0.1% to about 12%, in an embodiment from about 0.2% to about 5%, in a further embodiment from about 0.5% to about 4%, and in yet a further embodiment from about 1.0% to about 3.0%.

Structure of identified sebum modifier via differential scanning calorimetry experiment, example listed in table 1, is passed through the material studio software to calculate the properties and identify the descriptors which are unique to these sebum modifiers resulting in decrease in melting/reducing viscosity of the sebum. It has been found that the following materials can serve as sebum modifiers, as they reduce overall melt temperature of sebum to values below 25° C. They exhibit the following physical properties:

Jurs_WNSA_3≥−8; and a.

Shadow_YZ≤18; b.

Jurs descriptors (Stanton and Jurs, Development and use of charged partial surface area structural descriptors in computer-assisted quantitative structure-property relationship studies, Anal. Chem., 1990, 62 (21), pp 2323-2329) combines molecular shape and molecular surface charge area (electronic) information to characterize molecules. The descriptors are calculated by mapping atomic partial charges on solvent-accessible surface areas of individual atoms.

Jurs_WNSA_3 is known as Surface-weighted charged partial negative surface areas referred herein as the Molecular charge surface area=(Jurs_PNSA_3)* solvent-accessible surface areas (SASA)/1000

Where Jurs_PNSA_3 is known as Atomic charge weighted negative surface area =sum of the product of solvent-accessible surface area (SASA)×partial charge for all negatively charged atoms and

Shadow_YZ is defined as area of the molecular shadow in the YZ plane. This descriptor helps to characterize the shape of the molecules. The descriptor is calculated by projecting the molecular surface on three mutually perpendicular planes, XY, YZ, and XZ (R. H. Rohrbaugh and P. C. Jurs, Anal. Chim. Acta, 1987, 199, 99). This descriptor depends not only on conformation but also on the orientation of the molecule. To calculate them, the molecules are first rotated to align the principal moments of inertia with the X, Y, and Z axes. These properties can be calculated using Material Studio software (http://accelrys.com/products/collaborative-science/biovia-materials-studio/).

These two descriptors are typically used by a chemist to identify similar materials which can perform the same benefit.

TABLE 1
Example of Sebum Modifiers

| | Endothermic | Endothermic | Endothermic | | | |
|---|---|---|---|---|---|---|
| | Temperature | Temperature | Temperature | | | Viscosity |
| Sebum | Peak 1 | Peak 2 | Peak 3 | Jurs_ | | (×10⁻⁴) |
| Modifier Material | (° C.) | (° C.) | (° C.) | WNSA_3 | Shadow_YZ | Pa · s |
| 1,3-Butanediol | −8.00 | | 21 | −6 | 19 | 550 |
| Isopropyl myristate | −11.00 | | 9 | −7 | 32 | 500 |
| 2-Hexyldecanol | 0.50 | | | −4 | 30 | 400 |
| Propylene glycol | −17.40 | 5.70 | | −5 | 18 | 520 |
| PEG-3 glyceryl | 0.35 | 7.80 | | | | 680 |
| Cocoate | | | | | | |
| PEG-7 glyceryl | 0.25 | 8.00 | | | | 690 |
| Cocoate | | | | | | |
| Isododecane | −21.50 | −1.4 | 10.24 | −0.61 | 21 | 400 |
| Isoparaffin C11-12 | −8.00 | 4.00 | | | | 510 |

In an embodiment of the present invention, the Molecular charge surface area may be −6. In a further embodiment, the Molecular charge surface area may be −4. In a further embodiment, the Shadow length may be greater than 25. In a further embodiment, the Shadow length may be greater than 30

### FORMULATIONS AND EXAMPLES

The following are non-limiting examples of the present invention. The examples are given solely for the purpose of illustration and are not to be construed as limitations of the present invention, as many variations thereof are possible without departing from the spirit and scope of the invention, which would be recognized by one of ordinary skill in the art.

### EXAMPLES

### Method of Making Pre-Emulsion

Making the emulsion comprising components below is to pre-emulsify the sebum modifier before their addition to the pre-wash composition. A non-limiting example of a method of making is provided below. All oil soluble components are mixed in a vessel. Heat may be applied to allow mixture to liquidify. All water-soluble components are mixed in a separate vessel and heated to about same temperature as the oil phase. The oil phase and aqueous phase are mixed under a high shear mixer (example, Turrax mixer by IKA).

In an embodiment of the present invention, a pre-wash composition may comprise from about 0.25% to about 95% of a pre-emulsified emulsion, in a further embodiment from about 0.5% to about 95% of a pre-emulsified emulsion.

### Method of Making Aqueous Pre-Wash Compositions

The Sepigel 305 is then added to the pre-emulsion, if needed, and the solution is mixed using a high-speed-mixer for 2-5 minutes at 1800-2300 rpm until a uniform soluble composition is obtained.

### Method of Preparing Sebum-fluorescence Dye

An amount of 20 g of artificial sebum is mixed with 0.03 g of Tinopal B (Benzoxazole, 2,2′-(2,5-thiophenediyl) bis[5-(1,1-dimethylethyl), from BASF). The mixture is prepared in a brown vial to prevent light exposure and heated to 54 deg C. using water bath to melt and mix the components.

Artificial Sebum Composition: is prepared by adding materials mentioned in below table and then heated to 54 deg C. using water bath, to make it a homogenous mixture.

| | | Amount added |
|---|---|---|
| Raw Material | Weight % | (for 100 g) |
| Stearic Acid | 14 | 14 |
| Oleic acid | 8 | 8 |
| Squalene | 12 | 12 |
| Cetyl Palmitate | 12 | 12 |
| Isostearyl Isostearate | 12 | 12 |
| Trioctanoin | 20 | 20 |
| Caprylic Capric Triglyceride | 20 | 20 |
| Cholesterol | 2 | 2 |
| Total | 100 | Total |

Method of Treating Hair with Aqueous Pre-Wash Composition

An amount of 0.20 g of the Sebum-fluorescence dye mixture is applied and massaged onto hair switch onto natural virgin brown hair switches weighing 4.0 g via a syringe (dosage 0.05 g of sebum-fluorescence per g of hair). The hair images are taken under 256 nm UV light. Aqueous pre-wash composition is then applied on hair switch and left on hair switch under 25 deg C. and 50% relative humidity for 30 minutes. Then, the hair is wetted with water and 0.4 g of shampoo is applied (dosage 0.1 g of shampoo per g of hair). The shampoo is massaged into the hair for 10 seconds and rinsed with deionized water for 10 seconds. The hair Switch is then allowed to air dry and images are taken using camera under short wavelength UV light. The hair switch in this case is also assessed by expert graders, as described below, in addition to the Image analysis.

### Determination of Sebum Removal

The hair switches are treated with sebum-fluorescence dye and then the pre-wash composition is applied. The image of the hair switches is acquired using a digital single-lens reflex Camera with parallel polarizers immediately after applying the sebum (t₀) under 256 nm UV light. Another image is taken after washing the hair (t_{w}). The area occupied by blue color intensity light due to sebum-fluorescence mixture is analyzed (selecting the entire hair switch) using 2D projection (using Java-based image processing program). Then, the mean projected area is determined for the hair switch at t₀ (Aₜ₀) and for the hair at t_{w} (A_{rw}) and the sebum removal calculated using the equation given below. Each experiment is repeated with 3 hair switches. The percent sebum removal is calculated using below equation:

% Sebum Removal=100×(A_{tw}/Aₜ₀)

The standard error of sebum removal is less than 0.1

### Hair Switch Clean Appearance and Feel Assessment Method

The treated hair switches are air dried and rated by ten expert graders in terms of clean (non-greasy) appearance and feel based on a 5-point scale, 5 being the best clean (no greasy) and 1 being the worst clean (very greasy).

TABLE 1
Examples of Aqueous Pre-Wash Treatment Compositions

| | Pre-Wash | | | | | | |
|---|---|---|---|---|---|---|---|
| | treatment | | | | | | |
| Examples | Control | I | II | III | IV | V | VI |
| Raw Material | (wt./wt.) | (wt./wt.) | (wt./wt.) | (wt./wt.) | (wt./wt.) | (wt./wt.) | (wt./wt.) |
| | % | % | % | % | % | % | % |
| Distilled Water | QS | QS | QS | QS | QS | QS | QS |
| Polyacrylamide & C13-14 | 0.75 | 0.75 | 0.75 | 0.75 | 0.75 | 0.75 | 0.75 |
| Isoparaffin & Laureth-7 | | | | | | | |
| (Sepigel 305) | | | | | | | |
| Perfume | 0.2 | 0.2 | 0.2 | 0.2 | 0.2 | 0.2 | 0.2 |
| Isododecane | 0 | 1.0 | 0 | 0 | 0 | 0 | 0 |
| 2-Hexyl-1-decanol | 0 | 0 | 1.0 | 0 | 0 | 0 | 0 |
| 1,6 hexanediol | 0 | 0 | 0 | 0 | 1.0 | 0 | 0 |
| Glycolic acid | 0 | 0 | 0 | 0 | 0 | 1.0 | 0 |
| 4-hydroxy cinnamic acid | 0 | 0 | 0 | 0 | 0 | 0 | 1.0 |
| Oleic acid | 0 | 0 | 0 | 1.0 | 0 | 0 | 0 |
| Laureth-7 | 0.2 | 0.2 | 0.2 | 0.2 | 0.2 | 0.2 | 0.2 |
| PEG-100 Stearate | 0.2 | 0.2 | 0.2 | 0.2 | 0.2 | 0.2 | 0.2 |
| Cetearyl Glucoside & | 0.2 | 0.2 | 0.2 | 0.2 | 0.2 | 0.2 | 0.2 |
| Cetearyl Alcohol | | | | | | | |
| Methyl Paraben | 0.2 | 0.2 | 0.2 | 0.2 | 0.2 | 0.2 | 0.2 |
| Propyl Paraben | 0.15 | 0.15 | 0.15 | 0.15 | 0.15 | 0.15 | 0.15 |
| Benzyl Alcohol | 0.40 | 0.40 | 0.40 | 0.40 | 0.40 | 0.40 | 0.40 |
| PhenoxyEthanol | 0.40 | 0.40 | 0.40 | 0.40 | 0.40 | 0.40 | 0.40 |
| % Sebum Removal at dose | 78 | 90 | 88 | 86 | 88 | 75 | 72 |
| of 0.10 g of composition | | | | | | | |
| for 1.0 g of hair | | | | | | | |
| Clean Feel Rating at dose | 2.0 | 3.5 | 3.0 | 2.0 | 3.0 | 2.0 | 2.0 |
| of 0.10 g of composition for | | | | | | | |
| 1.0 g of hair (on 5 scale | | | | | | | |
| point with 5 as highest | | | | | | | |
| clean feel and 1 as lowest) | | | | | | | |
| Clean Look Rating at dose | 2.0 | 3.8 | 3.2 | 2.5 | 3.0 | 2.0 | 2.0 |
| of 0.10 g of composition for | | | | | | | |
| 1.0 g of hair (on 5 scale | | | | | | | |
| point with 5 as highest | | | | | | | |
| clean look and 1 as lowest) | | | | | | | |

Results: Formula I to IV showed increase in % sebum removal than control pre-wash treatment. Higher % sebum removal is observed in hair treated with higher doses of leave-on Formulas I-IV.

### Emulsifiers

Emulsifiers selection for each conditioning active is guided by the Hydrophilic-Lipophilic-Balance value (HLB value) of emulsifiers. Suitable range of HLB value is 6-16, more preferably 8-14. Emulsifiers with an HLB higher than 10 are water soluble. Emulsifiers with low HLB are lipid soluble. To obtain suitable HLB value, a mixture of two or more emulsifiers may be used. Suitable emulsifiers include non-ionic, cationic, anionic and amphoteric emulsifiers.

The concentration of the emulsifier in the composition should be sufficient to provide the desired emulsification of the conditioning actives to achieve desired particle sizes and emulsion stability, and generally ranges from about 0.1 wt. %-about 5 wt. %, from about 0.5 wt. %-about 3 wt. %, from about 0.6 wt. %-about 2 wt. %, for example.

Non-ionic emulsifiers suitable for use in the emulsion may include a wide variety of emulsifiers useful herein and include, but not limited to, those selected from the group consisting of sorbitan esters, glyceryl esters, polyglyceryl esters, methyl glucose esters, sucrose esters, ethoxylated fatty alcohols, hydrogenated castor oil ethoxylates, sorbitan ester ethoxylates, polymeric emulsifiers, and silicone emulsifiers.

Other suitable emulsifiers for use in the present invention include, but is not limited to, glyceryl monoesters, preferably glyceryl monoesters of C16-C22 saturated, unsaturated and branched chain fatty acids such as glyceryl oleate, glyceryl monostearate, glyceryl monopalmitate, glyceryl monobehenate, and mixtures thereof; polyglyceryl esters of C16-C22 saturated, unsaturated and branched-chain fatty acids, such as polyglyceryl-4 isostearate, polyglyceryl-3 oleate, diglycerol monooleate, tetraglycerol monooleate and mixtures thereof; methyl glucose esters, preferably methyl glucose esters of C16-C22 saturated, unsaturated and branched-chain fatty acids such as methyl glucose dioleate, methyl glucose sesquiisostearate, and mixtures thereof; sucrose fatty acid esters, preferably sucrose esters of C12-C22 saturated, unsaturated and branched-chain fatty acids such as sucrose stearate, sucrose trilaurate, sucrose distearate (e.g., Crodesta(Registered trademark) F10), and mixtures thereof; C12-C22 ethoxylated fatty alcohols such as oleth-2, oleth-3, steareth-2, and mixtures thereof; hydrogenated castor oil ethoxylates such as PEG-7 hydrogenated castor oil; sorbitan ester ethoxylates such as PEG-40 sorbitan peroleate, Polysorbate-80, and mixtures thereof; polymeric emulsifiers such as ethoxylated dodecyl glycol copolymer; and silicone emulsifiers such as laurylmethicone copolyol, cetyldimethicone, dimethicone copolyol, and mixtures thereof.

Anionic emulsifiers are suitable for use in the emulsion of the present invention. A variety of anionic emulsifiers can be used in the hair care composition as described herein. The anionic emulsifiers include, by way of illustrating and not limitation, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, gum arabic, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates.

In addition, anionic emulsifiers that have acrylate functionality useful herein include, but aren't limited to: poly(meth)acrylic acid; copolymers of (meth)acrylic acids and its (meth)acrylates with C1-22 alkyl, C1-C8 alkyl, butyl; copolymers of (meth)acrylic acids and (meth)acrylamide; Carboxyvinylpolymer; acrylate copolymers such as Acrylate/C10-30 alkyl acrylate crosspolymer, Acrylic acid/vinyl ester copolymer/Acrylates/Vinyl Isodecanoate crosspolymer, Acrylates/Palmeth-25 Acrylate copolymer, Acrylate/Steareth-20 Itaconate copolymer, and Acrylate/Celeth-20 Itaconate copolymer; Polystyrene sulphonate, copolymers of methacrylic acid and acrylamidomethylpropane sulfonic acid, and copolymers of acrylic acid and acrylamidomethylpropane sulfonic acid; carboxymethycellulose; carboxy guar; copolymers of ethylene and maleic acid; and acrylate silicone polymer. Neutralizing agents may be included to neutralize the anionic emulsifiers herein. Non-limiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof. Commercially available anionic emulsifiers include, for example, Carbomer supplied from Noveon under the tradename Carbopol 981 and Carbopol 980; Acrylates/C10-30 Alkyl Acrylate Crosspolymer having tradenames Pemulen TR-1, Pemulen TR-2, Carbopol 1342, Carbopol 1382, and Carbopol ETD 2020, all available from Noveon; sodium carboxymethylcellulose supplied from Hercules as CMC series; and Acrylate copolymer having a tradename Capigel supplied from Seppic. In another embodiment, anionic emulsifiers are carboxymethylcelluloses.

Cationic Emulsifers suitable for use in the emulsion of the present invention may include a wide variety of emulsifiers useful herein and include, but not limited to,: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt.

The cationic emulsifier is included in the composition at a level by weight of from about 0.1% to about 10%, preferably from about 0.5% to about 8%, more preferably from about 0.8% to about 5%, still more preferably from about 1.0% to about 4%.

### Mono-Long Alkyl Quaternized Ammonium Salt

The monoalkyl quaternized ammonium salt cationic surfactants useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

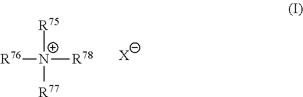

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

### Di-Long Alkyl Quaternized Ammonium Salt

Di-long alkyl quaternized ammonium salt is preferably combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of preferably from about 10% to about 50%, more preferably from about 30% to about 45%.

The dialkyl quaternized ammonium salt cationic surfactants useful herein are those having two long alkyl chains having 12-30 carbon atoms, preferably 16-24 carbon atoms, more preferably 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

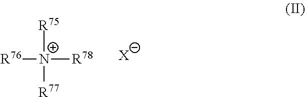

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof. Such dialkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants

Amphoteric emulsifiers suitable for use in the emulsion may include a wide variety of emulsifiers useful herein and include, but not limited to those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present hair care composition include cocoamphoacetate, cocoamphodiacetate, lauroamphoacetate, lauroamphodiacetate, and mixtures thereof.

In addition to these primary emulsifiers, the composition of the present invention can optionally contain a coemulsifier to provide additional water-lipid emulsion stability. Suitable coemulsifiers include, but are not limited to, phosphatidyl cholines and phosphatidyl choline-containing compositions such as lecithins; long chain C16-C22 fatty acid salts such as sodium stearate; long chain C16-C22 dialiphatic, short chain C1-C4 dialiphatic quaternary ammonium salts such as ditallow dimethyl ammonium chloride and ditallow dimethyl ammonium methylsulfate; long chain C16-C22 dialkoyl(alkenoyl)-2-hydroxyethyl, short chain C1-C4 dialiphatic quaternary ammonium salts such as ditallowoyl-2-hydroxyethyl dimethyl ammonium chloride; the long chain C16-C22 dialiphatic imidazolinium quaternary ammonium salts such as methyl-1-tallow amido ethyl-2-tallow imidazolinium methylsulfate and methyl-1-oleyl amido ethyl-2-oleyl imidazolinium methylsulfate; short chain C1-C4 dialiphatic, long chain C16-C22 monoaliphatic benzyl quaternary ammonium salts such as dimethyl stearyl benzyl ammonium chloride, and synthetic phospholipids such as stearamidopropyl PG-dimonium chloride (Phospholipid PTS from Mona Industries).

### Thickener

In one embodiment, the aqueous pre-wash hair care composition comprises a thickener to increase the substantivity and stability as well as ease of use (non-dripping) of the composition. Any suitable thickener can be used. In an embodiment, the aqueous pre-wash hair care composition may comprise from about 0.05% to about 10% of a thickener modifier, in a further embodiment, from about 0.1% to about 10% of a thickener, in yet a further embodiment, from about 0.5% to about 2% of a thickener, in a further embodiment, from about 0.7% to about 2% of a thickener, and in a further embodiment from about 1% to about 1.5% of a thickener. Nonlimited examples of such thickeners are modified silicas, fumed silicas, organoclays, waxes (hydrocarbon, silicone, fluoro-substituted, fatty acid esters), hydrophobically modified polysaccharides, hydrophobically modified polyurethanes or mixtures thereof.

The composition may also comprise at least one thickener. Nonlimited examples of such thickeners are modified silicas, fumed silicas, organoclays, waxes (hydrocarbon, silicone, fluoro-substituted, fatty acid esters), hydrophobically modified polysaccharides, hydrophobically modified polyurethanes or mixtures thereof.

### Carrier

The composition contains volatile or non-volatile, linear or branched hydrocarbon liquids or mixtures thereof. Non-limited examples include mineral oil, dodecane, isododecane, squalene, cholesterol, hydrogenated polyisobutylene, docosane, hexadecane, isohexadecane, other isoparaffins or mi,

The composition may comprise other volatile or non-volatile hydrophobic materials such as esters, ethers, carboxylic acids, esters, silicone oils, fatty alcohols, fatty amides,

In a further embodiment, the solvents may be dermatologically acceptable. In another embodiment, organic and silicone solvents that have boiling points below or equal to 250° C. may be volatile solvents and volatile carriers. In one embodiment, solvents with boiling points above 250° C. may be considered non-volatile.

### Silicones

The conditioning agent of the compositions of the present invention can be a silicone conditioning agent. The silicone conditioning agent may comprise volatile silicone, non-volatile silicone, or combinations thereof. The concentration of the silicone conditioning agent typically ranges from about 0.01% to about 10%, by weight of the composition, from about 0.1% to about 8%, from about 0.1% to about 5%, and/or from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609, which descriptions are incorporated herein by reference. The silicone conditioning agents for use in the compositions of the present invention can have a viscosity, as measured at 25° C., from about 20 to about 2,000,000 centistokes (“csk”), from about 1,000 to about 1,800,000 csk, from about 50,000 to about 1,500,000 csk, and/or from about 100,000 to about 1,500,000 csk.

The dispersed silicone conditioning agent particles typically have a volume average particle diameter ranging from about 0.01 micrometer to about 50 micrometer. For small particle application to hair, the volume average particle diameters typically range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, and/or from about 20 micrometer to about 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

### Organic Conditioning Materials

The conditioning agent of the compositions of the present invention may also comprise at least one organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

### Hair Health Actives

In an embodiment of the present invention, a scalp health active may be added to provide scalp benefits. This group of materials is varied and provides a wide range of benefits including anti-dandruff, anti-fungal, anti-microbial, moisturization, barrier improvement, and anti-oxidant, anti-itch, and sensates. Such skin health actives include but are not limited to: zinc pyrithione, climbazole, octopirox, vitamin E and F, salicylic acid, glycols, glycolic acid, PCA, PEGs, erythritol, glycerin, lactates, hyaluronates, allantoin and other ureas, betaines, sorbitol, glutamates, xylitols, menthol, menthyl lactate, isocyclomone, benzyl alcohol, and natural extracts/oils including peppermint, spearmint, argan, jojoba and aloe.

### Anti-Dandruff Actives

In an embodiment of the present invention, the compositions may contain anti-dandruff actives and anti-fungal and antimicrobial actives. When present in these compositions, the anti-dandruff, anti-fungal and antimicrobial active is typically included in an amount of about 0.01 wt. % to about 5 wt. %, based on the total weight of the pre-wash composition. In these compositions, the active should be physically and chemically compatible with other ingredients of the composition, and should not otherwise unduly impair product stability, aesthetics, or performance. Anti-dandruff actives suitable for use in the pre-wash compositions include pyridinethione salts, azoles, selenium sulfide, particulate sulfur, salicylic acid, and mixtures thereof. A typical anti-dandruff active is pyridinethione salt, such as zinc pyrithione (ZPT). Pre-wash compositions can also include a zinc-containing layered material. An example of a zinc-containing layered material can include zinc carbonate materials. Of these, zinc carbonate and pyridinethione salts (particularly zinc pyridinethione or “ZPT) are common in the composition, and often present together. Non-limiting examples of azole actives are benzimidazole, benzothiazole, bifonazole, butaconazole nitrate, climbazole, itraconazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. The pre-wash compositions may also include one or more anti-fungal or anti-microbial actives. Suitable anti-microbial actives include coal tar, sulfur, charcoal, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-hydroxyquinoline, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof.

### Optional Ingredients

The compositions of the present invention can also additionally comprise any suitable optional ingredients as desired. For example, the composition can optionally include other active or inactive ingredients.

The compositions may include other common hair ingredients such as other anti-dandruff actives, minoxidil, conditioning agents, and other suitable materials. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein. Examples of these ingredient classes include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, propellants, reducing agents, sequestrants, rheology modifiers, hair conditioning agents, and surfactants.

The formulations of the present invention may be present in typical hair care compositions. They may be in the form of solutions, dispersion, emulsions, powders, talcs, encapsulated, spheres, spongers, solid dosage forms, foams, and other delivery mechanisms. The composition of the present invention may be hair tonics, leave-on hair products such as conditioners, treatment, and styling products, and any other form that may be applied to the hair.

### ADDITIONAL EXAMPLES/COMBINATIONS

A. A pre-wash composition comprising:
   a) from about 0.1% to about 12.0% of a sebum modifier material or mixture of sebum modifier materials, wherein the sebum modifier material has the following physical properties:
      i. Molecular charge surface area higher than −8; and
      ii. Shadow length greater than 18.
   b) from about 88 wt. % to about 99.9 wt. % water carrier;
   c) from about 0.1 wt. % to about 5 wt. % emulsifier selected from the group consisting of anionic, non-ionic, cationic and amphoteric.
B) A pre-wash composition according to Paragraph A, wherein the sebum modifier material is selected from the group consisting of 1,3-butanediol, Isopropyl myristate, 2-hexyldecanol, propylene glycol, PEG-3 glyceryl Cocoate, PEG-7 glyceryl cocoate, Isododecane and Isoparaffin C11-12 and mixtures thereof.
C) A pre-wash composition according to Paragraph A-B, wherein the sebum modifier material is from about 0.2% to 5%
D) A pre-wash composition according to Paragraph A-C, wherein the sebum modifier material is from about 0.5% to about 4%.
E) A pre-wash composition according to Paragraph A-D, wherein the sebum modifier material is from about 1% to about 3%.
F) A pre-wash composition according to Paragraph A-E, wherein the emulsifier is from about 0.5 wt. % to about 3 wt. %.
G) A pre-wash composition according to Paragraph A-F, wherein the emulsifier is from about 0.6 wt. %-about 2 wt. %.
H) A pre-wash composition according to Paragraph A-G, wherein the emulsifier is an anionic emulsifier selected from the group consisting of water-soluble salts of alkyl sulfates, alkyl ether sulfates, alkyl isothionates, alkyl carboxylates, alkyl sulfosuccinates, alkyl succinamates, alkyl sulfate salts, sodium dodecyl sulphate, phospholipids or lecithin, or soaps, sodium, potassium or ammonium stearate, oleate, palmitate, alkylarylsulfonic acid salts, isobutylene-maleic anhydride copolymer, gum arabic, sodium alginate, carboxymethylcellulose, cellulose sulfate and pectin, poly(styrene sulfonate), isobutylene-maleic anhydride copolymer, gum arabic, carrageenan, sodium alginate, pectic acid, tragacanth gum, almond gum and agar; semi-synthetic polymers, synthetic polymers, polyacrylic acid, polymethacrylic acid, acrylic acid butyl acrylate copolymer or crotonic acid homopolymers and copolymers, vinylbenzenesulfonic acid, 2-acrylamido-2-methylpropanesulfonic acid homopolymers and copolymers, carboxymodified polyvinyl alcohol, sulfonic acid-modified polyvinyl alcohol, phosporic acid-modified polyvinyl alcohol, phosphate, sulfated tristyrylphenol ethoxylates, poly(meth)acrylic acid; copolymers of (meth)acrylic acids and its (meth)acrylates with C1-22 alkyl, C1-C8 alkyl, butyl,copolymers of (meth)acrylic acids and (meth)acrylamide, Carboxyvinylpolymer; acrylate copolymers, polystyrene sulphonate, copolymers of methacrylic acid and acrylamidomethylpropane sulfonic acid, copolymers of acrylic acid and acrylamidomethylpropane sulfonic acid, carboxymethycellulose; carboxy guar; copolymers of ethylene and maleic acid, acrylate silicone polymer and mixtures thereof.
I) A pre-wash composition according to Paragraph A-H, wherein the emulsifier is an nonionic emulsifier selected from the group consisting of sorbitan esters, glyceryl esters, polyglyceryl esters, methyl glucose esters, sucrose esters, ethoxylated fatty alcohols, hydrogenated castor oil ethoxylates, sorbitan ester ethoxylates, polymeric emulsifiers, silicone emulsifiers and mixtures thereof.
J) A pre-wash composition according to Paragraph A-I, wherein the emulsifier is an cationic emulsifier selected from the group consisting of mono-long alkyl quaternized ammonium salt, di-long quarternized ammonium salt, a combination of mono-long alkyl quaternized ammonium salt and di-long alkyl quaternized ammonium salt, mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt and mixtures thereof.
K) A pre-wash composition according to Paragraph A-J, wherein the emulsifier is an amphoteric emulsifier selected from the group consisting of derivatives of aliphatic secondary and tertiary amines in which the aliphatic radical can be straight or branched chain and wherein one of the aliphatic substituents contains from about 8 to about 18 carbon atoms and one contains an anionic group, cocoamphoacetate, cocoamphodiacetate, lauroamphoacetate, lauroamphodiacetate, and mixtures thereof.
L) A pre-wash composition according to Paragraph A-K, wherein the emulsifier is selected from the group consisting of PEG-100 stearate, laureth-7 and mixtures thereof.
M) A cleansing method comprising the steps of
   a. Applying on hair a pre-wash composition comprising according to A-L:
      1) from about 0.1% to about 12.0% of a sebum modifier material or mixture of sebum modifier materials wherein the sebum modifier material has the following physical properties:
         (i) Molecular charge surface area higher than −8; and
         (ii) Shadow length greater than 18.
      2) from about 88 wt. % to about 99.9 wt. % water carrier;
      3) from about 0.1 wt. % to about 30 wt. % emulsifier selected from the group consisting of anionic, non-ionic, cationic and amphoteric
   b. spreading the pre-shampoo on hair and scalp;
   c. optionally rinsing the pre-wash composition from hair and scalp with water;
   d. applying a shampoo composition on hair comprising from about 5 wt. % to about 40 wt. % of detersive surfactant;
   e. spreading the shampoo composition on hair and scalp;
   f. rinsing the shampoo composition from hair and scalp with water wherein the cleansing method removes from about 10 wt. % to about 60 wt. % more sebum compared to a cleansing method that does not include steps a to c.
N) A method according to Paragraph A-M wherein the cleansing method removes from about 15 wt. % to about 40 wt. % more sebum compared to a cleansing method that does not include steps a to c.
0) A method according to Paragraph A-N, wherein the cleansing method removes from about 15 wt. % to about 30 wt. % more sebum compared to a cleansing method that does not include steps a to c.
P) A method according to Paragraph A-O, wherein the sebum modifier material is selected from the group consisting of 1,3-butanediol, Isopropyl myristate, 2-hexyldecanol, propylene glycol, PEG-3 glyceryl Cocoate, PEG-7 glyceryl cocoate, Isododecane and Isoparaffin C11-12 and mixtures thereof.
Q) A method according to Paragraph A-P, wherein the sebum modifier material is from about 0.2% to 5%.
R) A method according to Paragraph A-Q, wherein the sebum modifier material is from about 0.5% to about 4%.
S) A pre-wash composition according to Paragraph A-R, wherein the sebum modifier material is from about 1% to about 3%.
T) A pre-wash composition according to Paragraph A-S, wherein the emulsifier is from about 0.5 wt. % to about 3 wt. %.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of Embodiments of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A pre-wash composition comprising:
a) from about 0.1% to about 12.0% of a sebum modifier material or mixture of sebum modifier materials, wherein the sebum modifier material has the following physical properties:
i. Molecular charge surface area higher than −8; and
ii. Shadow length greater than 18.
b) from about 88 wt. % to about 99.9 wt. % water carrier;
c) from about 0.1 wt. % to about 5 wt. % emulsifier selected from the group consisting of anionic, non-ionic, cationic and amphoteric.

2. A pre-wash composition according to claim 1 wherein the sebum modifier material is selected from the group consisting of 1,3-butanediol, Isopropyl myristate, 2-hexyldecanol, propylene glycol, PEG-3 glyceryl Cocoate, PEG-7 glyceryl cocoate, Isododecane and Isoparaffin C11-12 and mixtures thereof.

3. A pre-wash composition according to claim 1 wherein the sebum modifier material is from about 0.2% to 5%.

4. A pre-wash composition according to claim 1 wherein the sebum modifier material is from about 0.5% to about 4%.

5. A pre-wash composition according to claim 1 wherein the sebum modifier material is from about 1% to about 3%.

6. A pre-wash composition according to claim 1 wherein the emulsifier is from about 0.5 wt. % to about 3 wt. %.

7. A pre-wash composition according to claim 1 wherein the emulsifier is from about 0.6 wt. %-about 2 wt. %.

8. A pre-wash composition according to claim 1 wherein the emulsifier is an anionic emulsifier selected from the group consisting of water-soluble salts of alkyl sulfates, alkyl ether sulfates, alkyl isothionates, alkyl carboxylates, alkyl sulfosuccinates, alkyl succinamates, alkyl sulfate salts, sodium dodecyl sulphate, phospholipids or lecithin, or soaps, sodium, potassium or ammonium stearate, oleate, palmitate, alkylarylsulfonic acid salts, isobutylene-maleic anhydride copolymer, gum arabic, sodium alginate, carboxymethylcellulose, cellulose sulfate and pectin, poly(styrene sulfonate), isobutylene-maleic anhydride copolymer, gum arabic, carrageenan, sodium alginate, pectic acid, tragacanth gum, almond gum and agar; semi-synthetic polymers, synthetic polymers, polyacrylic acid, polymethacrylic acid, acrylic acid butyl acrylate copolymer or crotonic acid homopolymers and copolymers, vinylbenzenesulfonic acid, 2-acrylamido-2-methylpropanesulfonic acid homopolymers and copolymers, carboxymodified polyvinyl alcohol, sulfonic acid-modified polyvinyl alcohol, phosporic acid-modified polyvinyl alcohol, phosphate, sulfated tristyrylphenol ethoxylates, poly(meth)acrylic acid; copolymers of (meth)acrylic acids and its (meth)acrylates with C1-22 alkyl, C1-C8 alkyl, butyl,copolymers of (meth)acrylic acids and (meth)acrylamide, Carboxyvinylpolymer;
acrylate copolymers, polystyrene sulphonate, copolymers of methacrylic acid and acrylamidomethylpropane sulfonic acid, copolymers of acrylic acid and acrylamidomethylpropane sulfonic acid, carboxymethycellulose; carboxy guar; copolymers of ethylene and maleic acid, acrylate silicone polymer and mixtures thereof.

9. A pre-wash composition according to claim 1 wherein the emulsifier is an nonionic emulsifier selected from the group consisting of sorbitan esters, glyceryl esters, polyglyceryl esters, methyl glucose esters, sucrose esters, ethoxylated fatty alcohols, hydrogenated castor oil ethoxylates, sorbitan ester ethoxylates, polymeric emulsifiers, silicone emulsifiers and mixtures thereof.

10. A pre-wash composition according to claim 1 wherein the emulsifier is an cationic emulsifier selected from the group consisting of mono-long alkyl quaternized ammonium salt, di-long quarternized ammonium salt, a combination of mono-long alkyl quaternized ammonium salt and di-long alkyl quaternized ammonium salt, mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt and mixtures thereof.

11. A pre-wash composition according to claim 1 wherein the emulsifier is an amphoteric emulsifier selected from the group consisting of derivatives of aliphatic secondary and tertiary amines in which the aliphatic radical can be straight or branched chain and wherein one of the aliphatic substituents contains from about 8 to about 18 carbon atoms and one contains an anionic group, cocoamphoacetate, cocoamphodiacetate, lauroamphoacetate, lauroamphodiacetate, and mixtures thereof.

12. A pre-wash composition according to claim 1 wherein the emulsifier is selected from the group consisting of PEG-100 stearate, laureth-7 and mixtures thereof.

13. A cleansing method comprising the steps of
a. Applying on hair a pre-wash composition comprising:
1. from about 0.1% to about 12.0% of a sebum modifier material or mixture of sebum modifier materials wherein the sebum modifier material has the following physical properties:
(i) Molecular charge surface area higher than −8; and
(ii) Shadow length greater than 18.
2. from about 88 wt. % to about 99.9 wt. % water carrier;
3. from about 0.1 wt. % to about 30 wt. % emulsifier selected from the group consisting of anionic, non-ionic, cationic and amphoteric.
b. spreading the pre-shampoo on hair and scalp;
c. optionally rinsing the pre-wash composition from hair and scalp with water;
d. applying a shampoo composition on hair comprising from about 5 wt. % to about 40 wt. % of detersive surfactant;
e. spreading the shampoo composition on hair and scalp;
f. rinsing the shampoo composition from hair and scalp with water wherein the cleansing method removes from about 10 wt. % to about 60 wt. % more sebum compared to a cleansing method that does not include steps a to c.

14. A method according to claim 13 wherein the cleansing method removes from about 15 wt. % to about 40 wt. % more sebum compared to a cleansing method that does not include steps a to c.

15. A method according to claim 13 wherein the cleansing method removes from about 15 wt. % to about 30 wt. % more sebum compared to a cleansing method that does not include steps a to c.

16. A method according to claim 13 wherein the sebum modifier material is selected from the group consisting of 1,3-butanediol, Isopropyl myristate, 2-hexyldecanol, propylene glycol, PEG-3 glyceryl Cocoate, PEG-7 glyceryl cocoate, Isododecane and Isoparaffin C11-12 and mixtures thereof.

17. A method according to claim 13 wherein the sebum modifier material is from about 0.2% to 5%.

18. A method according to claim 13 wherein the sebum modifier material is from about 0.5% to about 4%.

19. A pre-wash composition according to claim 13 wherein the sebum modifier material is from about 1% to about 3%.

20. A pre-wash composition according to claim 13 wherein the emulsifier is from about 0.5 wt. % to about 3 wt. %.
